(19) United States
(12) Patent Application Publication
(10) Pub. No.: US 2007/0292918 A1
(43) Pub. Date: Dec. 20, 2007

(54) Codon optimization method

(76) Inventors: Steven J. Stelman, Santee, CA (US); Douglas Hershberger, San Diego, CA (US); Tom M. Ramseier, Newton, MA (US)

Correspondence Address: TRASK BRITT, P.O. BOX 2550, SALT LAKE CITY, UT, 84110, US

(21) Appl. No.: 11/809,010

(22) Filed: May 30, 2007

Related U.S. Application Data
(60) Provisional application No. 60/901,687, filed on Feb. 14, 2007.
(60) Provisional application No. 60/809,536, filed on May 30, 2006.

(51) Int. Cl. C12P 21/06; G01N 33/48

(52) U.S. Cl. 435/69.1; 702/20

(57) ABSTRACT

A heterologous expression in a host Pseudomonas bacteria of an optimized polynucleotide sequence encoding a protein.

### CROSS REFERENCE TO RELATED APPLICATIONS

This application claims priority to U.S. Provisional Application Ser. Nos. 60/901,687, filed Feb. 14, 2007, and 60/809,536, filed May 30, 2006.

### FIELD OF THE INVENTION

The present invention relates generally to methods for optimizing genes for bacterial expression. The invention further relates to a database system and tools for analysis of optimized genes.

### BACKGROUND OF THE INVENTION

Numerous bacteria have been used as host cells for the preparation of heterologous recombinant proteins. One significant disadvantage of numerous bacterial systems is their use of rare codons, which is very different from the codon preference in human genes. The presence of these rare codons can lead to delayed and reduced expression of recombinant genes. In certain aspects, a nucleic acid sequence may be modified to encode a recombinant polypeptide variant wherein specific codons of the nucleic acid sequence have been changed to codons that are favored by a particular host and can result in enhanced levels of expression (see, e.g., Haas et al., Curr. Biol. 6:315, 1996; Yang et al., Nucleic Acids Res. 24:4592, 1996).

The process of optimizing the nucleotide sequence coding for a heterologously expressed protein can be an important step for improving expression yields. The optimization requirements may include steps to improve the ability of the host to produce the foreign protein as well as steps to assist the researcher in efficiently designing expression constructs. Although prices for gene-scale DNA synthesis have declined significantly in recent years, the investment in the synthesis of an optimized gene for this purpose can be costly. Therefore, it is important that a thorough analysis be conducted to ensure that all design requirements have been properly satisfied before proceeding with synthesis. Furthermore, the process of assessing candidate synthetic genes and producing human-readable reports of the results of this analysis is a time consuming process.

Although several tools exist for the calculation of codon preference, these tools are not generally designed to report codon usage in a usable context. As these tools do not compare a calculated usage with a reference standard, manual reformatting of the output data is typically required in order to distinguish the presence of rare codons relative to the host expression system. Spatial visualization of rare codons along the translated gene sequence must also be performed manually. Thus, substantial user training, including importing the desired sequence into the correct format for each application, is required.

### BRIEF SUMMARY OF THE INVENTION

The present invention includes a synthetic polynucleotide sequence that has been optimized for heterologous expression in a bacterial host cell such as Pseudomonas fluorescens.

The present invention also provides a method of producing a recombinant protein in the cytoplasm and periplasm of the bacterial cell including optimizing a synthetic polynucleotide sequence for heterologous expression in a bacterial host, wherein the synthetic polynucleotide comprises a nucleotide sequence encoding a protein, such as an antigen. The method also includes ligating the optimized synthetic polynucleotide sequence into an expression vector and transforming the host bacteria with the expression vector. The method additionally includes culturing the transformed host bacteria in a suitable culture media appropriate for the expression of the protein and isolating the protein. The bacteria host selected can be Pseudomonas fluorescens.

Other embodiments of the present invention include methods of optimizing synthetic polynucleotide sequences for heterologous expression in a host cell by identifying and modifying rare codons from the synthetic polynucleotide sequence that are rarely used in the host. Furthermore, these methods can include identification and modification of putative internal ribosomal binding site sequences as well as identification and modification of extended repeats of G or C nucleotides from the synthetic polynucleotide sequence. The methods can also include identification and minimization of protective antigen protein secondary structures in the RBS and gene coding regions, as well as modifying undesirable enzyme-restriction sites from the synthetic polynucleotide sequences.

The present invention also provides automatic serial analysis and report generation of a gene using a database and tools to calculate codon usage from a raw sequence and graphically report the location of the rare codons along a translated DNA sequence. Where multiple candidate versions of a particular gene are designed, an analysis of all versions is performed to determine the best candidate for synthesis. This comparison, along with a comparison of the candidate versions with that of a reference codon preference, is presented in a useful human-readable format.

### BRIEF DESCRIPTION OF THE SEVERAL VIEWS OF THE DRAWINGS

FIG. 1 illustrates a flow diagram showing steps that can be used during optimization of a synthetic polynucleotide sequence;

FIGS. 2 and 3 illustrate rare codon usage profiles showing the location and distribution of rare codons along a translated protein sequence in P. fluorescens strain MB214; and

FIG. 4 illustrates an embodiment of a database schema for the gene database of the present invention.

### DETAILED DESCRIPTION OF THE INVENTION

The present invention is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

The invention generally relates to a process for preparing a heterologous recombinant protein in a prokaryotic host cell. The codon use of the host cell for host cell genes is determined. Rarely occurring codons are modified with frequently occurring codons in the nucleic acid coding for the heterologous recombinant protein in the host cell. The host cell is then transformed with the nucleic acid coding for the recombinant protein and the recombinant nucleic acid is expressed.

As used herein, the terms “modify” or “alter”, or any forms thereof, mean to modify, alter, replace, delete, substitute, remove, vary, or transform.

The present invention also relates to synthetic polynucleotide sequences that encode for a protein. Embodiments of the present invention also provide for the heterologous expression of a synthetic polynucleotide in a bacterial host. Other embodiments include a heterologous expression of a synthetic polynucleotide in Pseudomonas fluorescens. Additional embodiments of the present invention also include optimized polynucleotide sequences encoding a recombinant protein that can be expressed using a heterologous Pseudomonas fluorescens-based expression system. Another embodiment of the present invention also includes a heterologous expression of a synthetic polynucleotide in the cytoplasm of Pseudomonas fluorescens. Additional embodiment of the present invention also includes a heterologous expression of a synthetic polynucleotide in the periplasm of Pseudomonas fluorescens.

In heterologous expression systems, optimization steps may improve the ability of the host to produce the foreign protein. Protein expression is governed by a host of factors including those that affect transcription, mRNA processing, and stability and initiation of translation. The polynucleotide optimization steps may include steps to improve the ability of the host to produce the foreign protein as well as steps to assist the researcher in efficiently designing expression constructs. Optimization strategies may include, for example, the modification of translation initiation regions, alteration of mRNA structural elements, and the use of different codon biases. The following paragraphs discuss potential problems that may result in reduced heterologous protein expression, and techniques that may overcome these problems.

One area that can result in reduced heterologous protein expression is a rare codon-induced translational pause. A rare codon-induced translational pause includes the presence of codons in the polynucleotide of interest that are rarely used in the host organism may have a negative effect on protein translation due to their scarcity in the available tRNA pool. One method of improving optimal translation in the host organism includes performing codon optimization which can result in rare host codons being modified in the synthetic polynucleotide sequence.

Another area that can result in reduced heterologous protein expression is by alternate translational initiation. Alternate translational initiation can include a synthetic polynucleotide sequence inadvertently containing motifs capable of functioning as a ribosome binding site (RBS). These sites can result in initiating translation of a truncated protein from a gene-internal site. One method of reducing the possibility of producing a truncated protein, which can be difficult to remove during purification, includes modifying putative internal RBS sequences from an optimized polynucleotide sequence.

Another area that can result in reduced heterologous protein expression is through repeat-induced polymerase slippage. Repeat-induced polymerase slippage involves nucleotide sequence repeats that have been shown to cause slippage or stuttering of DNA polymerase which can result in frameshift mutations. Such repeats can also cause slippage of RNA polymerase. In an organism with a high G+C content bias, there can be a higher degree of repeats composed of G or C nucleotide repeats. Therefore, one method of reducing the possibility of inducing RNA polymerase slippage includes altering extended repeats of G or C nucleotides.

Another area that can result in reduced heterologous protein expression is through interfering secondary structures. Secondary structures can sequester the RBS sequence or initiation codon and have been correlated to a reduction in protein expression. Stemloop structures can also be involved in transcriptional pausing and attenuation. An optimized polynucleotide sequence can contain minimal secondary structures in the RBS and gene coding regions of the nucleotide sequence to allow for improved transcription and translation.

Another area that can effect heterologous protein expression are restriction sites: By modifying restriction sites that could interfere with subsequent sub-cloning of transcription units into host expression vectors a polynucleotide sequence can be optimized.

Optimizing a DNA sequence can negatively or positively affect gene expression or protein production. For example, modifying a less-common codon with a more common codon may affect the half life of the mRNA or alter its structure by introducing a secondary structure that interferes with translation of the message. It may therefore be necessary, in certain instances, to alter the optimized message.

All or a portion of a gene can be optimized. In some cases the desired modulation of expression is achieved by optimizing essentially the entire gene. In other cases, the desired modulation will be achieved by optimizing part but not all of the gene.

The codon usage of any coding sequence can be adjusted to achieve a desired property, for example high levels of expression in a specific cell type. The starting point for such an optimization may be a coding sequence with 100% common codons, or a coding sequence which contains a mixture of common and non-common codons.

Two or more candidate sequences that differ in their codon usage can be generated and tested to determine if they possess the desired property. Candidate sequences can be evaluated by using a computer to search for the presence of regulatory elements, such as silencers or enhancers, and to search for the presence of regions of coding sequence which could be converted into such regulatory elements by an alteration in codon usage. Additional criteria may include enrichment for particular nucleotides, e.g., A, C, G or U, codon bias for a particular amino acid, or the presence or absence of particular mRNA secondary or tertiary structure. Adjustment to the candidate sequence can be made based on a number of such criteria.

Promising candidate sequences are constructed and then evaluated experimentally. Multiple candidates may be evaluated independently of each other, or the process can be iterative, either by using the most promising candidate as a new starting point, or by combining regions of two or more candidates to produce a novel hybrid. Further rounds of modification and evaluation can be included.

Modifying the codon usage of a candidate sequence can result in the creation or destruction of either a positive or negative element. In general, a positive element refers to any element whose alteration or removal from the candidate sequence could result in a decrease in expression of the therapeutic protein, or whose creation could result in an increase in expression of a therapeutic protein. For example, a positive element can include an enhancer, a promoter, a downstream promoter element, a DNA binding site for a positive regulator (e.g., a transcriptional activator), or a sequence responsible for imparting or modifying an mRNA secondary or tertiary structure. A negative element refers to any element whose alteration or removal from the candidate sequence could result in an increase in expression of the therapeutic protein, or whose creation would result in a decrease in expression of the therapeutic protein. A negative element includes a silencer, a DNA binding site for a negative regulator (e.g., a transcriptional repressor), a transcriptional pause site, or a sequence that is responsible for imparting or modifying an mRNA secondary or tertiary structure. In general, a negative element arises more frequently than a positive element. Thus, any change in codon usage that results in an increase in protein expression is more likely to have arisen from the destruction of a negative element rather than the creation of a positive element. In addition, alteration of the candidate sequence is more likely to destroy a positive element than create a positive element. In one embodiment, a candidate sequence is chosen and modified so as to increase the production of a therapeutic protein. The candidate sequence can be modified, e.g., by sequentially altering the codons or by randomly altering the codons in the candidate sequence. A modified candidate sequence is then evaluated by determining the level of expression of the resulting therapeutic protein or by evaluating another parameter, e.g., a parameter correlated to the level of expression. A candidate sequence which produces an increased level of a therapeutic protein as compared to an unaltered candidate sequence is chosen.

In another approach, one or a group of codons can be modified, e.g., without reference to protein or message structure and tested. Alternatively, one or more codons can be chosen on a message-level property, e.g., location in a region of predetermined, e.g., high or low GC content, location in a region having a structure such as an enhancer or silencer, location in a region that can be modified to introduce a structure such as an enhancer or silencer, location in a region having, or predicted to have, secondary or tertiary structure, e.g., intra-chain pairing, inter-chain pairing, location in a region lacking, or predicted to lack, secondary or tertiary structure, e.g., intra-chain or inter-chain pairing. A particular modified region is chosen if it produces the desired result.

Methods which systematically generate candidate sequences are useful. For example, one or a group, e.g., a contiguous block of codons, at various positions of a synthetic nucleic acid sequence can be modified with common codons (or with non common codons, if for example, the starting sequence has been optimized) and the resulting sequence evaluated. Candidates can be generated by optimizing (or de-optimizing) a given “window” of codons in the sequence to generate a first candidate, and then moving the window to a new position in the sequence, and optimizing (or de-optimizing) the codons in the new position under the window to provide a second candidate. Candidates can be evaluated by determining the level of expression they provide, or by evaluating another parameter, e.g., a parameter correlated to the level of expression. Some parameters can be evaluated by inspection or computationally, e.g., the possession or lack thereof of high or low GC content; a sequence element such as an enhancer or silencer; secondary or tertiary structure, e.g., intra-chain or inter-chain paring.

In certain embodiments, the optimized nucleic acid sequence can express its protein, at a level which is at least 110%, 150%, 200%, 500%, 1,000%, 5,000% or even 10,000% of that expressed by nucleic acid sequence that has not been optimized

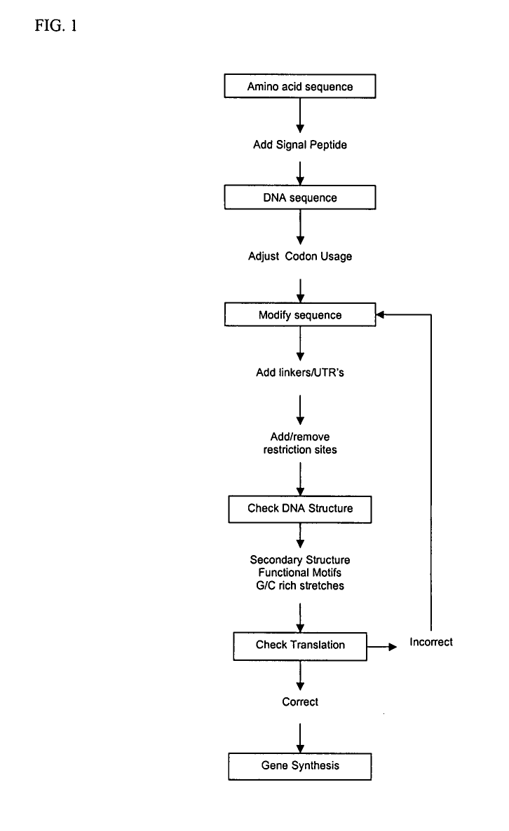

As illustrated by FIG. 1, the optimization process can begin by identifying the desired amino acid sequence to be heterologously expressed by the host. From the amino acid sequence a candidate polynucleotide or DNA sequence can be designed. During the design of the synthetic DNA sequence, the frequency of codon usage can be compared to the codon usage of the host expression organism and rare host codons can be modified in the synthetic sequence. Additionally, the synthetic candidate DNA sequence can be modified in order to remove undesirable enzyme restriction sites and add or alter any desired signal sequences, linkers or untranslated regions. The synthetic DNA sequence can be analyzed for the presence of secondary structure that may interfere with the translation process, such as G/C repeats and stem-loop structures. Before the candidate DNA sequence is synthesized, the optimized sequence design can be checked to verify that the sequence correctly encodes the desired amino acid sequence. Finally, the candidate DNA sequence can be synthesized using DNA synthesis techniques, such as those known in the art.

In another embodiment of the invention, the general codon usage in a host organism, such as Pseudomonas fluorescens, can be utilized to optimize the expression of the heterologous polynucleotide sequence. The percentage and distribution of codons that rarely would be considered as preferred for a particular amino acid in the host expression system can be evaluated. Values of 5% and 10% usage can be used as cutoff values for the determination of rare codons. For example, the codons listed in TABLE 1 have a calculated occurrence of less than 5% in the Pseudomonas fluorescens MB214 genome and would be generally avoided in an optimized gene expressed in a Pseudomonas fluorescens host.

TABLE 1

| Amino Acid(s) | Codon(s) Used | % Occurrence |
|---|---|---|
| G Gly | GGA | 3.26 |
| I Ile | ATA | 3.05 |
| L Leu | CTA | 1.78 |
| | CTT | 4.57 |
| | TTA | 1.89 |
| R Arg | AGA | 1.39 |
| | AGG | 2.72 |
| | CGA | 4.99 |
| S Ser | TCT | 4.18 |

A variety of host cells can be used for expression of a desired heterologous gene product. The host cell can be selected from an appropriate population of E. coli cells or Psuedomonas cells. Pseudomonads and closely related bacteria, as used herein, is co-extensive with the group defined herein as “Gram(-) Proteobacteria Subgroup 1.” “Gram(-) Proteobacteria Subgroup 1” is more specifically defined as the group of Proteobacteria belonging to the families and/or genera described as falling within that taxonomic “Part” named “Gram-Negative Aerobic Rods and Cocci” by R. E. Buchanan and N. E. Gibbons (eds.), Bergey's Manual of Determinative Bacteriology, pp. 217-289 (8th ed., 1974) (The Williams & Wilkins Co., Baltimore, Md., USA) (hereinafter “Bergey (1974)”). The host cell can be selected from Gram-negative Proteobacteria Subgroup 18, which is defined as the group of all subspecies, varieties, strains, and other sub-special units of the species Pseudomonas fluorescens, including those belonging, e.g., to the following (with the ATCC or other deposit numbers of exemplary strain(s) shown in parenthesis): P. fluorescens biotype A, also called biovar 1 or biovar I (ATCC 13525); P. fluorescens biotype B, also called biovar 2 or biovar II (ATCC 17816); P. fluorescens biotype C, also called biovar 3 or biovar III (ATCC 17400); P. fluorescens biotype F, also called biovar 4 or biovar IV (ATCC 12983); P. fluorescens biotype G, also called biovar 5 or biovar V (ATCC 17518); P. fluorescens biovar VI; P. fluorescens Pf0-1; P. fluorescens Pf-5 (ATCC BAA-477); P. fluorescens SBW25; and P. fluorescens subsp. cellulosa (NCIMB 10462).

The host cell can be selected from Gram-negative Proteobacteria Subgroup 19, which is defined as the group of all strains of P. fluorescens biotype A, including P. fluorescens strain MB101, and derivatives thereof.

In one embodiment, the host cell can be any of the Proteobacteria of the order Pseudomonadales. In a particular embodiment, the host cell can be any of the Proteobacteria of the family Pseudomonadaceae. In a particular embodiment, the host cell can be selected from one or more of the following: Gram-negative Proteobacteria Subgroup 1, 2, 3, 5, 7, 12, 15, 17, 18 or 19.

Additional P. fluorescens strains that can be used in the present invention include P. fluorescens Migula and P. fluorescens Loitokitok, having the following ATCC designations: [NCIB 8286]; NRRL B-1244; NCIB 8865 strain COI; NCIB 8866 strain CO2; 1291 [ATCC 17458; IFO 15837; NCIB 8917; LA; NRRL B-1864; pyrrolidine; PW2 [ICMP 3966; NCPPB 967; NRRL B-899]; 13475; NCTC 10038; NRRL B-1603 [6; IFO 15840]; 52-1C; CCEB 488-A [BU 140]; CCEB 553 [IEM 15/47]; IAM 1008 [AHH-27]; IAM 1055 [AHH-23]; 1 [IFO 15842]; 12 [ATCC 25323; NIH 11; den Dooren de Jong 216]; 18 [IFO 15833; WRRL P-7]; 93 [TR-10]; 108[52-22; IFO 15832]; 143 [IFO 15836; PL]; 149 [2-40-40; IFO 15838]; 182 [IFO 3081; PJ 73]; 184 [IFO 15830]; 185-[W2 L-1]; 186 [IFO 15829; PJ 79]; 187 [NCPPB 263]; 188 [NCPPB 316]; 189 [PJ227; 1208]; 191 [IFO 15834; PJ 236; 22/1]; 194 [Klinge R-60; PJ 253]; 196 [PJ 288]; 197 [PJ 290]; 198[PJ 302]; 201 [PJ 368]; 202 [PJ 372]; 203 [PJ 376]; 204 [IFO 15835; PJ 682]; 205[PJ686]; 206 [PJ 692]; 207 [PJ 693]; 208 [PJ 722]; 212 [PJ 832]; 215 [PJ 849]; 216 [PJ885]; 267 [B-9]; 271 [B-1612]; 401 [C71A; IFO 15831; PJ 187]; NRRL B-3178 [4; IFO 15841]; KY8521; 3081; 30-21; [IFO 3081]; N; PYR; PW; D946-B83 [BU 2183; FERM-P 3328]; P-2563 [FERM-P 2894; IFO 3658]; IAM-1126 [43F]; M-1; A506 [A5-06]; A505-[A5-05-1]; A526 [A5-26]; B69; 72; NRRL B4290; PMW6 [NCIB 11615]; SC 12936; A1 [IFO 15839]; F 1847 [CDC-EB]; F 1848 [CDC 93]; NCIB 10586; P17; F-12; AmMS 257; PRA25; 6133D02; 6519E01; Ni; SC15208; BNL-WVC; NCTC 2583 [NCIB 8194]; H13; 1013 [ATCC 11251; CCEB 295]; IFO 3903; 1062; or Pf-5.

Transformation of the Pseudomonas host cells with the vector(s) may be performed using any transformation methodology known in the art, and the bacterial host cells may be transformed as intact cells or as protoplasts (i.e. including cytoplasts). Transformation methodologies include poration methodologies, e.g., electroporation, protoplast fusion, bacterial conjugation, and divalent cation treatment, e.g., calcium chloride treatment or CaCl/Mg²⁺ treatment, or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

As used herein, the term “fermentation” includes both embodiments in which literal fermentation is employed and embodiments in which other, non-fermentative culture modes are employed. Fermentation may be performed at any scale. In embodiments of the present invention the fermentation medium can be selected from among rich media, minimal media, and mineral salts media; a rich medium can also be used. In another embodiment either a minimal medium or a mineral salts medium is selected. In still another embodiment, a minimal medium is selected. In yet another embodiment, a mineral salts medium is selected. Mineral salts media are generally used.

Mineral salts media consists of mineral salts and a carbon source such as, e.g., glucose, sucrose, or glycerol. Examples of mineral salts media include, e.g., M9 medium, Pseudomonas medium (ATCC 179), Davis and Mingioli medium (see, BD Davis & ES Mingioli (1950) in J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. No organic nitrogen source, such as peptone, tryptone, amino acids, or a yeast extract, is included in a mineral salts medium. Instead, an inorganic nitrogen source is used and this may be selected from among, e.g., ammonium salts, aqueous ammonia, and gaseous ammonia. A mineral salts medium can contain glucose as the carbon source. In comparison to mineral salts media, minimal media can also contain mineral salts and a carbon source, but can be supplemented with, e.g., low levels of amino acids, vitamins, peptones, or other ingredients, though these are added at very minimal levels.

In one embodiment, media can be prepared using the various components listed below. The components can be added in the following order: first (NH₄)HPO₄, KH₂PO₄ and citric acid can be dissolved in approximately 30 liters of distilled water; then a solution of trace elements can be added, followed by the addition of an antifoam agent, such as Ucolub N 115. Then, after heat sterilization (such as at approximately 121 degree. C.), sterile solutions of glucose MgSO₄ and thiamine-HCL can be added. Control of pH at approximately 6.8 can be achieved using aqueous ammonia. Sterile distilled water can then be added to adjust the initial volume to 371 minus the glycerol stock (123 mL). The chemicals are commercially available from various suppliers, such as Merck. This media can allow for a high cell density cultivation (HCDC) for growth of Pseudomonas species and related bacteria. The HCDC can start as a batch process which is followed by a two-phase fed-batch cultivation. After unlimited growth in the batch part, growth can be controlled at a reduced specific growth rate over a period of 3 doubling times in which the biomass concentration can increased several fold. Further details of such cultivation procedures is described by Riesenberg, D.; Schulz, V.; Knorre, W. A.; Pohl, H. D.; Korz, D.; Sanders, E. A.; Ross, A.; Deckwer, W. D. (1991) “High cell density cultivation of. Escherichia coli, at controlled specific growth rate” J Biotechnol: 20(1) 17-27. TABLE-US-00005 TABLE 5 Medium composition Component Initial concentration KH₂PO₄ 13.3 gl⁻¹ (NH₄) 2HPO₄4.0 g l⁻¹ Citric acid 1.7 g l⁻¹ MgSO₄-7H₂O 1.2 g l⁻¹ Trace metal solution 10 mll⁻¹ Thiamin HCl 4.5 mg l⁻¹ Glucose-H₂O 27.3 g l⁻¹ Antifoam Ucolub N115 0.1 ml l⁻¹ Feeding solution MgSO₄-7H₂O 19.7 g l⁻¹ Glucose-H₂O 770 g l⁻¹ NH₃ 23 g Trace metal solution 6 g l⁻¹ Fe(111) citrate 1.5 g l⁻¹ MnCl₂-4H₂O 0.84 g l⁻¹ ZmCH₂COOl₂-2H₂O 0.3 g l⁻¹ H₃BO₃ 0.25 g l⁻¹ Na₂MoO₄-2H₂O 0.25 g l⁻¹ CoCl₂ 6H₂O 0.15 g l⁻¹ CuCl₂ 2H₂O 0.84 g l⁻¹ ethylene diaminetetracetic acid Na₂ salt 2H₂O (Titriplex III, Merck).

The sequences recited in this application may be homologous (have similar identity). Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. For example, any naturally occurring nucleic acid can be modified by any available mutagenesis method to include one or more selector codon. When expressed, this mutagenized nucleic acid encodes a polypeptide comprising one or more unnatural amino acid. The mutation process can, of course, additionally alter one or more standard codon, thereby changing one or more standard amino acid in the resulting mutant protein as well. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95%, 96%, 97%, 98% or 99% or more can also be used to establish homology. Methods for determining sequence similarity percentages (e.g., BLASTP and BLASTN using default parameters) are described herein and are generally available.

Polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345 358; Hein J. (1990) Unified Approach to Alignment and Phylogenes pp. 626 645 Methods in Enzymology vol. 183, Academic Press, Inc., San Diego, Calif.; Higgins, D. G. and Sharp, P. M. (1989) CABIOS 5:151 153; Myers, E. W. and Muller W. (1988) CABIOS 4:11 17; Robinson, E. D. (1971) Comb. Theor 11:105; Santou, N. Nes, M. (1987) Mol. Biol. Evol. 4:406 425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726 730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that can be suitable for determining percent sequence identity and sequence similarity are the BLAST and BLAST 2.0 algorithms, which are described in Altschul et al. (1977) Nucl. Acids Res. 25:3389 3402 and Altschul et al. (1990) J. Mol. Biol. 215:403 410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, codon optimized sequences can include a polypeptide which may be a fusion polypeptide that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide, allowing the production of increased levels, relative to a non-fused polypeptide, in an expression system. Briefly, nucleic acid sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46, 1985; Murphy et al., Proc. Natl. Acad. Sci. USA 83:8258 8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The present invention also provides automatic serial analysis and report generation of a gene using a database and tools to calculate codon usage from a raw sequence and graphically report the location of the rare codons along a translated DNA sequence. Several new tools have been developed to assist in this process, wherein analysis and report generation are completed automatically, reducing the required time spent by a researcher.

In the initial stages of project design, a protein's coding sequence can be evaluated to determine if optimization of all or part of the gene is advisable. While there is no absolute criterion in making this determination, one strategy involves evaluation of the percentage and distribution of codons that would be considered rarely preferred for a particular amino acid in the host expression system. Values of 5% and 10% usage are commonly used as cutoff values for the determination of rare codons. For example, the codons listed in Table 1 have a calculated occurrence of less than 5% in the MB214 genome, and would be preferentially avoided in an optimized gene to be expressed in that host. To ascertain whether a gene of interest might be expressed heterologously without optimization, one may determine what percentage of rare codons exist in that gene and whether they reside in locations that could have a deleterious effect on expression (i.e. near the 5′ end of the gene or concentrated together into clusters).

To address these issues, the tool of the present invention is designed to calculate codon usage from a raw ORF sequence and to graphically report the location of the rare codons along a translated DNA sequence. Additionally, a color-coded table can be presented to compare the codon usage of the submitted gene with that of the MB214 reference codon preference. In order to allow portability, remove dependence on any particular underlying bioinformatics package and provide ease of use, the new tool can be written as a CGI program entirely in the Perl programming language, and be accessible as a form via a web browser.

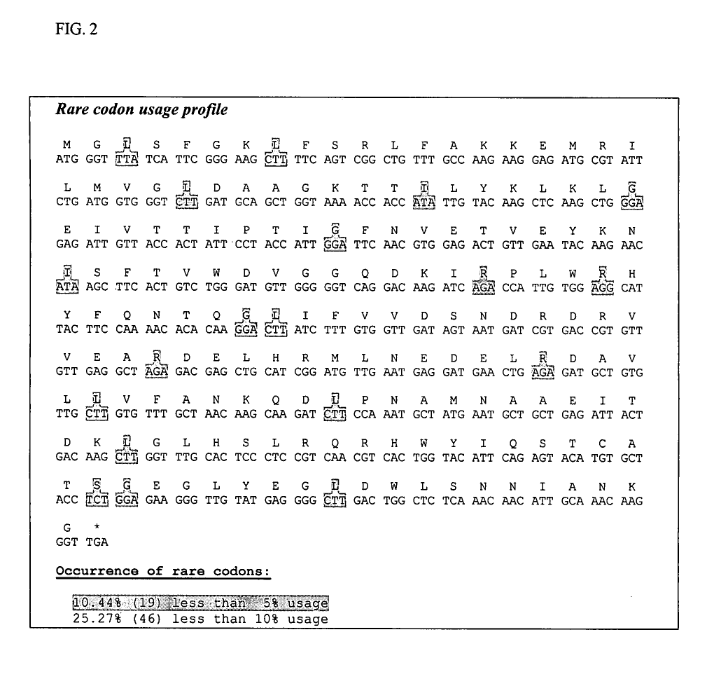

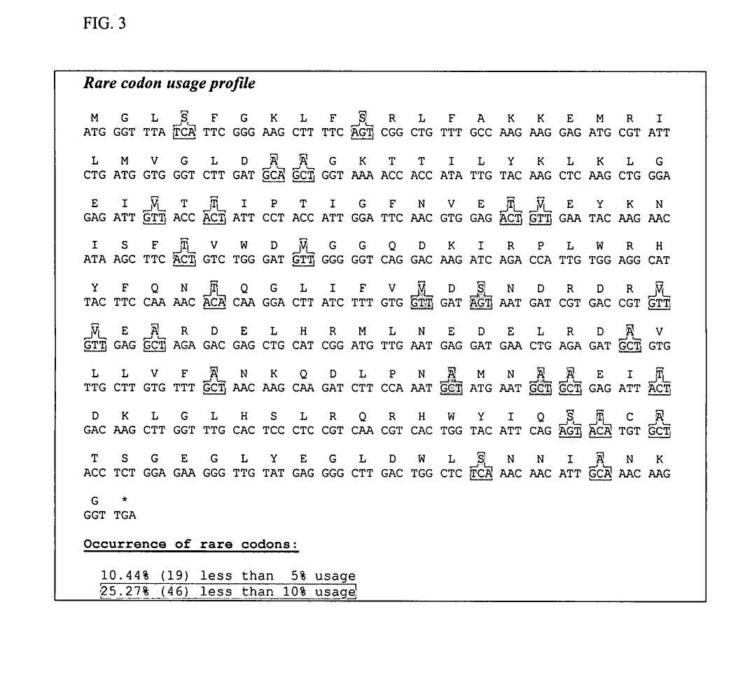

In use, a non-formatted nucleotide sequence is pasted into the form and submitted, and formatted reports are returned. Sample results are shown in FIGS. 2 and 3, and Table 2.

TABLE 2

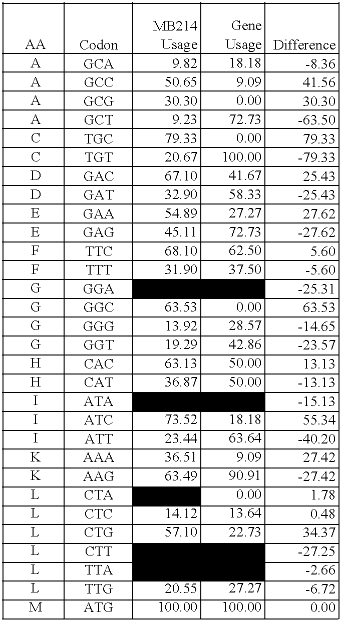

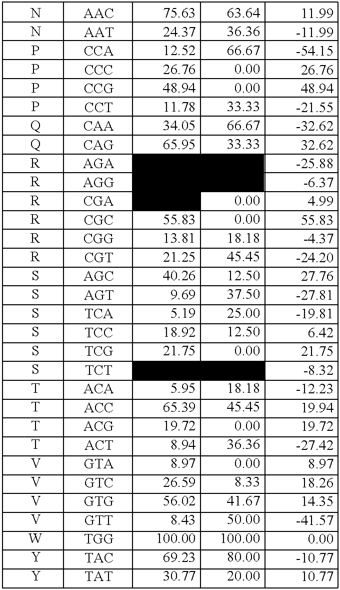

Table 2 represents a codon frequency table, listing for each amino acid/codon pair: i) the percent frequency of the codon in MB214, ii) the percent frequency of the codon in the analyzed gene, and iii) the percent difference between the usage in the analyzed gene versus M1214. Highlighting indicates codon usage in MB214 of less than 10%. Highlighting of “0.00” values in the Gene Usage column indicates a rare codon that is not used in the analyzed sequence.

FIGS. 2 and 3 illustrate results of rare codon usage profiles showing the location and distribution of rare codons along a translated protein sequence. Highlighted codons are represented with less than 5% and 10% frequency in P. fluorescens strain MB214 in FIGS. 2 and 3, respectively. The overall percentage and absolute number of codons falling below 5% or 10% usage is also indicated following the translated sequence in FIGS. 2 and 3, respectively.

Database and tools for analysis of optimized genes are also provided. Once a gene has been analyzed and a determination made that synthesis of an optimized version of the gene is warranted, one or more synthetic versions of the gene can be designed. The resulting gene design candidates can each be analyzed prior to synthesis to ensure compliance with all design criteria. In order to keep track of submitted genes, associated design criteria, and the resulting synthetic candidate versions to be analyzed, a relational database is provided to store this information.

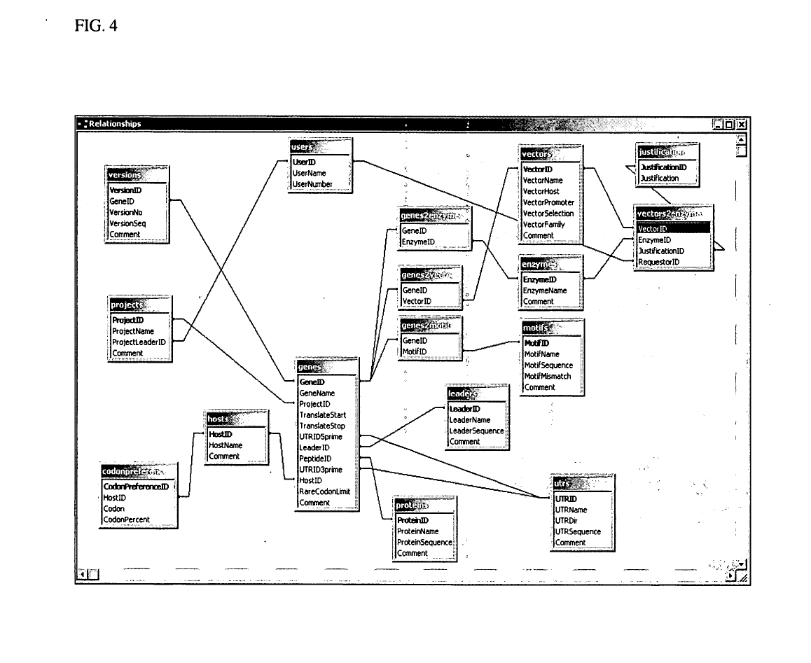

In order to function with existing Perl code in a Linux environment, in a particular embodiment of the invention, PostgreSQL was selected as the relational database. Data can be entered into and extracted from the created database using, for example, Perl's DBI module. The database schema can be designed to allow flexibility in selecting elements to be included in the synthetic transcription unit (e.g., protein sequence, leader sequence, and UTR's). Expression vectors and hosts can be defined to ensure compatibility of the synthetic gene with vector multiple cloning sites and host codon preferences. Motifs that should be avoided in the final sequence can also be defined, and candidate synthetic versions for each gene can be stored. A representative embodiment of the database schema for the gene database is illustrated in FIG. 4, with filed names in the actual database represented in lower case.

In order to facilitate entry of data into the database without requiring expertise in SQL, in a particular embodiment of the invention, a user interface was developed consisting of CGI generated HTML forms. The user interface can also provide a layer of error checking to make sure all entered values are valid.

Entering a new gene requires completed CGI-generated HTML form and pressing a SUBMIT button. Values may either be entered into the form freely in text boxes or selected from pre-defined pull-down and check box menus. These menus can be built automatically from values currently available in the database. New values can be added for each menu by clicking a respective “Add” hyperlink, which spawns a new HTML form specific to that data entry. If errors are detected upon submission, the user can be returned to the form and presented with messages describing the necessary corrections that must be made. All previously entered values can be preserved on the form so that only the error-related values can be modified or re-entered.

After entering a new gene, a quote can be requested from an outside vendor for design and synthesis of the candidate gene/transcription unit. The process can be initiated by entering information onto the vendor's website page. In order to facilitate this process and to prevent data entry errors, a tool can be provided that allows preparation of the necessary data directly from the database into the required format. This tool can allow a user to generate the required information for a quote by selecting a gene name from an automatically generated pull-down menu of all genes available in the database at the time the page was loaded. Once a gene is selected, clicking a SUBMIT button generates a form with three fields that can be pasted directly into the vendor's quote request form. A hyperlink to this page can also be provided.

Due to redundancy in the genetic code, there are numerous different coding sequences that can be generated for a synthetic gene candidate. Vendors will typically provide multiple candidate synthetic versions for each gene in order to allow a researcher to select the version that most closely matches the required design criteria. These sequences can be added to the database and associated with the respective gene submission using the web. A gene name can then be selected from an automatically generated pull-down menu, and a version number, sequence, and any descriptive comments can be entered. Once submitted, the automated analysis pipeline can be run to determine which of the submitted versions in the database is most optimal for synthesis.

A program (e.g., a Perl program) can be included to automate the process of evaluating each candidate synthetic version to ensure compliance with design criteria as submitted to the database. Each synthetic gene version can be extracted from the database, along with the relevant design specifications, and run through a series of analyses. These analysis can include one or more of the following:
1) GCG (available from Accelrys Software, Inc., San Diego, Calif.) CODONFREQUENCY can be run to determine the codon usage of the synthetic version. Output files are parsed and the presence of any rare codons, defined by a percent cutoff value stored in the database for each gene, can be detected;
2) GCG MAPSORT can be run to determine the presence of any unwanted restriction enzymes that may interfere with future subcloning. The list of evaluated restriction enzymes can be extracted from the database through relationships between enzymes, expression vectors, and genes. Output files can be parsed to detect the presence of any restriction site from the list of enzymes;
3) GCG FINDPATTERNS can be run to detect the presence of any sequence motifs that should be avoided in the synthetic version. Each pattern can be defined in the database along with the number of tolerated mismatches for that specific pattern. Output files can be parsed to detect the presence of any of the defined deleterious sequence motifs;
4) A program (e.g., a Perl program) can be run to detect the strength of any stemloop structures present. The program can sequentially run GCG STEMLOOP to find locations of putative stemloops in the sequence, extract the coordinates of those loops, and then run the loop coordinates through GCG MFOLD to determine the free energy of the loop structure. Output results can be sorted by free energy and the data for the five strongest loops can be extracted. Additionally, the free energy of the strongest loop can be reported for comparative purposes; and
5) GCG BESTFIT can be run to compare the peptide translations of the native and synthetic DNA sequences to ensure no mutations have been introduced by error. Translated sequences can be generated by GCG TRANSLATE. Output results can be parsed and reported.

A report can be generated in HTML format for viewing or printing in a web browser or Microsoft Word. The report can include a summary report of the results of the analyses in tabular form. For example, as illustrated in Table 3, one column can be provided for each synthetic version and one row for each analysis.

TABLE 3

| Criteria | v1 | v2 | v3 |
|---|---|---|---|
| Rare Codons | | | |
| ≧5 G's or C's | | | |
| Gene-internal SD sequence | | | |
| Strongest gene-internal steploop structure | | | |
| Unique restriction sites | | | |
| Synthetic gene encoded protein is identical to the original | | | |
| protein sequence | | | |

In this manner, a researcher can compare the results for each version and select the most suitable version for synthesis. If analysis indicates that none of the versions meet the design criteria, additional versions can be requested and analysis can be rerun until a suitable version is obtained. The report can also include the raw data from each analysis for documentation purposes. Data for each gene version can be collated by analysis performed and relevant parts of the output data can be highlighted for ease of reading.

The present invention is explained in greater detail in the Examples that follow. These examples are intended as illustrative of the invention and are not to be taken are limiting thereof.

### EXAMPLES

### Example 1

### Design of Synthetic Gene from P. fluorescens

A DNA region containing an optimal Shine-Dalgamo sequence and a unique SpeI restriction enzyme site was added upstream of the coding sequence. A DNA region containing three stop codons and a unique XhoI restriction enzyme site was added downstream of the coding sequence. All rare codons occurring in the Pfenex ORFome with less than 5% codon usage were modified to avoid ribosomal stalling. All gene-internal ribosome binding sites which matched the pattern aggaggtn₅₋₁₀dtg with two or fewer mismatches were modified to avoid truncated protein products. Stretches of five or more C, or five or more G nucleotides were eliminated to avoid RNA polymerase slippage. Strong gene-internal stem-loop structures, especially ones covering the ribosome binding site, were modified. The synthetic gene was synthesized by DNA2.0, Inc. (Menlo Park, Calif.).

### Example 2

### Design of Synthetic Gene from P. fluorescens

The amino acids from methionine 21 to glutamine 520 were included in the final expressed protein product. All rare codons occurring in the Pfenex ORFome with less than 5% codon usage were modified to avoid ribosomal stalling. All gene-internal ribosome binding sites which matched the pattern aggaggtn₅₋₁₀dtg with two or fewer mismatches were modified to avoid truncated protein products. Stretches of five or more C or five or more G nucleotides were eliminated to avoid RNA polymerase slippage. Strong gene-internal stem-loop structures, especially ones covering the ribosome binding site, were modified. A DNA sequence encoding the 24 amino acid pbp periplasmic secretion leader was fused to the 5′ end of the optimized sequence. A DNA region containing an optimal Shine-Dalgamo sequence and a unique SpeI restriction enzyme site was added upstream of the coding sequence. A DNA region containing three stop codons and a unique XhoI restriction enzyme site was added downstream of the coding sequence. The synthetic gene was synthesized by DNA2.0, Inc.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A method of producing a recombinant protein comprising:
optimizing a synthetic polynucleotide sequence for heterologous expression in a host Pseudomonas fluorescens bacteria, wherein the synthetic polynucleotide comprises a nucleotide sequence encoding a protein;
ligating the optimized synthetic polynucleotide sequence into an expression vector;
transforming the host Pseudomonas fluorescens bacteria with the expression vector;
culturing the transformed host Pseudomonas fluorescens bacteria in a suitable culture media appropriate for the expression of the protein; and
isolating the protein.

2. The method of claim 1, wherein optimizing the synthetic polynucleotide sequence for heterologous expression in the host Pseudomonas fluorescens bacteria further comprises identifying and modifying rare codons from the synthetic polynucleotide sequence that are rarely used in the host Pseudomonas fluorescens bacteria.

3. The method of claim 2, wherein optimizing the synthetic polynucleotide sequence for heterologous expression in the host Pseudomonas fluorescens bacteria further comprises identifying and modifying putative internal ribosomal binding site sequences from the synthetic polynucleotide sequence.

4. The method of claim 2, wherein optimizing the synthetic polynucleotide sequence for heterologous expression in the host Pseudomonas fluorescens bacteria further comprises identifying and modifying extended repeats of G or C nucleotides from the synthetic polynucleotide sequence.

5. The method of claim 2, wherein optimizing the synthetic polynucleotide sequence for heterologous expression in the host Pseudomonas fluorescens bacteria further comprises identifying and minimizing mRNA secondary structure in the RBS and gene coding regions of the synthetic polynucleotide sequence.

6. The method of claim 2, wherein optimizing the synthetic polynucleotide sequence for heterologous expression in the host Pseudomonas fluorescens bacteria further comprises identifying and modifying undesirable enzyme-restriction sites from the synthetic polynucleotide sequence.

7. The method of claim 2, wherein identifying and modifying rare codons comprises identifying and modifying codons having an occurrence of less than 10% in the Pseudomonas fluorescens bacterial genome.

8. The method of claim 2, wherein identifying and modifying rare codons comprises identifying and modifying codons having an occurrence of less than 5% in the Pseudomonas fluorescens bacterial genome.

9. The method of claim 1, wherein optimizing the synthetic polynucleotide sequence for heterologous expression further comprises identifying and modifying codons from the synthetic polynucleotide sequence to increase expression.

10. The method of claim 2, wherein the modifying rare codons comprises replacing the rare codons with frequently occurring codons.

11. A method of producing a recombinant protein comprising:
identifying and modifying rare codons from the synthetic polynucleotide sequence that are rarely used in the host Pseudomonas bacteria;
identifying and modifying putative internal ribosomal binding site sequences from the synthetic polynucleotide sequence;
identifying and modifying extended repeats of G or C nucleotides from the synthetic polynucleotide sequence;
identifying and minimizing mRNA secondary structure in the RBS and gene coding regions of the synthetic polynucleotide sequence;
identifying and modifying undesirable enzyme-restriction sites from the synthetic polynucleotide sequence to form an optimized synthetic polynucleotide sequence;
ligating the optimized synthetic polynucleotide sequence into an expression vector;
transforming the host Pseudomonas bacteria with the expression vector;
culturing the transformed host Pseudomonas bacteria in a suitable culture media appropriate for the expression of the protein; and
isolating the protein.

12. The method of claim 11, wherein the host Pseudomonas bacteria is Pseudomonas fluorescens.

13. The method of claim 11, wherein the host Pseudomonas bacteria is Pseudomonas fluorescens strain MB 101.

14. The method of claim 12, wherein identifying and modifying rare codons comprises identifying and modifying codons having an occurrence of less than 10% in the Pseudomonas fluorescens bacterial genome.

15. The method of claim 12, wherein identifying and modifying rare codons comprises identifying and modifying codons having an occurrence of less than 5% in the Pseudomonas fluorescens bacterial genome.

16. A method of analyzing optimized genes, comprising:
providing a gene optimization database for Pseudomonas fluorescens bacteria;
entering gene data into the database;
identifying expression vectors or hosts;
submitting synthesis request of a candidate gene or transcription unit;
adding optimized gene sequences into the database;
evaluating one or more synthetic versions of synthesized candidate gene(s) to ensure compliance with synthesis request; and
analyzing the one or more synthetic versions of candidate gene(s).

17. The method of claim 16, further comprising generating a report of results from analysis of the one or more synthetic versions of candidate gene(s).

18. The method of claim 16, wherein analyzing the one or more synthetic versions of candidate gene(s) comprises analyzing candidate gene(s) by inspection or computationally.

19. The method of claim 16, wherein analyzing the one or more synthetic versions of candidate gene(s) comprises analyzing the level of expression provided by candidate gene(s).

20. The method of claim 16, wherein analyzing the one or more synthetic versions of candidate gene(s) comprises analyzing the possession or lack thereof of high or low GC content, a sequence element, or the structure of the candidate gene(s).


SEQUENCE LISTING (1 sequence)

SEQ ID NO: 1 - DNA, 20 bp, Pseudomonas fluorescens
  misc_feature (8)..(12): n can be A, T, G, C
  misc_feature (13)..(17): n is optional and can be A, T, G, C
aggaggtnnnnnnnnnndtg